(19) United States
(12) Patent Application Publication
(10) Pub. No.: US 2026/0061143 A1
(43) Pub. Date: Mar. 5, 2026

(54) MAINTAINING THERMAL ENERGY TISSUE SHRINKAGE AND RESTORING MECHANICAL PROPERTIES OF THE TISSUE VIA PROTEIN CROSSLINKER DELIVERY

(71) Applicant: Spinal Simplicity, LLC, Overland Park, KS (US)

(72) Inventors: Thomas P. Hedman, Lexington, KY (US); Adam Rogers, Olathe, KS (US); John Goebel, Asheville, NC (US)

(21) Appl. No.: 19/311,979

(22) Filed: Aug. 27, 2025

Related U.S. Application Data
(60) Provisional application No. 63/688,060, filed on Aug. 28, 2024.

(51) Int. Cl. A61M 5/46; A61B 18/00; A61B 18/12; A61B 18/14; A61B 18/16; A61K 31/352

(52) U.S. Cl. CPC A61M 5/46; A61B 18/148; A61B 18/16; A61K 31/352; A61B 2018/00714; A61B 2018/126; A61B 2018/162

(57) ABSTRACT

A method, kits, and devices for performing the method of immediately improving and/or restoring the mechanical integrity and material properties of thermally shrunk collagenous tissue through delivery of a non-toxic protein crosslinker to the tissue. A thermal energy device shrinks the target tissue using controlled application of thermal energy. A non-toxic protein crosslinker is injected and/or topically applied to the target tissue before, during, and/or after thermal shrinkage. A dual applicator may comprise a thermal energy probe and injection needle. The thermal energy probe may apply thermal energy to the target tissue and the injection needle may apply the non-toxic protein crosslinker to the target tissue. A kit for use in performing the method may provide various components used for applying thermal energy and applying the non-toxic protein crosslinker to the target tissue.

### RELATED APPLICATIONS

This patent application is a non-provisional application claiming priority benefit, with regard to all common subject matter, of U.S. Provisional Patent Application No. 63/688,060, filed Aug. 28, 2024, and entitled “MAINTAINING THERMAL ENERGY TISSUE SHRINKAGE AND RESTORING MECHANICAL PROPERTIES OF THE TISSUE VIA PROTEIN CROSSLINKER DELIVERY.” The above-referenced application is hereby incorporated by reference in its entirety into the present application.

This patent application shares certain subject matter in common with the following earlier-filed patents: U.S. patent application Ser. No. 10/230,671, filed Aug. 29, 2002, entitled USE OF NON-TOXIC CROSSLINKING REAGENTS TO IMPROVE FATIGUE RESISTANCE AND REDUCE MECHANICAL DEGRADATION OF INTERVERTEBRAL DISC AND OTHER COLLAGENOUS TISSUES, now U.S. Pat. No. 9,084,772; U.S. patent application Ser. No. 12/966,812, filed Aug. 11, 2015, entitled USE OF NON-TOXIC CROSSLINKING REAGENTS TO IMPROVE FATIGUE RESISTANCE AND REDUCE MECHANICAL DEGRADATION OF INTERVERTEBRAL DISC AND OTHER COLLAGENOUS TISSUES, now U.S. Pat. No. 9,101,602; U.S. patent application Ser. No. 10/786,861, filed Feb. 24, 2004, entitled NON-TOXIC CROSSLINKING REAGENTS TO RESIST CURVE PROGRESSION IN SCOLIOSIS AND INCREASE DISC PERMEABILITY, now U.S. Pat. No. 7,435,722; U.S. patent application Ser. No. 11/346,464, filed Feb. 2, 2006, entitled DIRECT APPLICATION OF NON-TOXIC CROSSLINKING REAGENTS TO RESIST PROGRESSIVE SPINAL DEFORMITY, now U.S. Pat. No. 8,153,600; U.S. patent application Ser. No. 11/712,684, filed Feb. 28, 2007, entitled METHOD OF TREATING A KNEE MENISCUS WITH A CROSS-LINKING REAGENT TO INCREASE RESISTANCE TO TEARING OR RUPTURING, now U.S. Pat. No. 8,022,101; U.S. patent application Ser. No. 12/715,737, filed Mar. 2, 2010, entitled DIRECT APPLICATION OF NON-TOXIC CROSSLINKING REAGENTS TO RESIST PROGRESSIVE SPINAL DEGENERATION AND DEFORMITY, now U.S. Pat. No. 8,211,938; U.S. patent application Ser. No. 14/178,523, filed Feb. 12, 2014, entitled CROSSLINKER ENHANCED REPAIR OF CONNECTIVE TISSUES, now U.S. Pat. No. 10,278,947; U.S. patent application Ser. No. 16/171,714, filed Oct. 26, 2018, entitled CROSSLINKER ENHANCED REPAIR OF CONNECTIVE TISSUES, now U.S. Pat. No. 10,980,771; U.S. patent application Ser. No. 12/816,674, filed Jun. 16, 2010, entitled NON-TOXIC CROSSLINKING REAGENTS TO RESIST CURVE PROGRESSION IN SCOLIOSIS AND INCREASE DISC PERMEABILITY, now U.S. Pat. No. 8,450,276; U.S. patent application Ser. No. 11/975,072, filed Oct. 17, 2007, entitled DIRECT APPLICATION OF NON-TOXIC CROSSLINKING REAGENTS TO RESIST PROGRESSIVE SPINAL DEGENERATION AND DEFORMITY, now U.S. Pat. No. 8,119,599; U.S. patent application Ser. No. 12/203,730, filed Sep. 3, 2008, entitled FORMULATIONS FOR NONSURGICAL EXOGENOUS CROSSLINK THERAPY, now U.S. Pat. No. 8,198,248; U.S. patent application Ser. No. 12/496,045, filed Jul. 1, 2009, entitled FORMULATIONS FOR NONSURGICAL EXOGENOUS CROSSLINK THERAPY, now U.S. Pat. No. 8,283,322; U.S. patent application Ser. No. 13/704,220, filed Dec. 13, 2012, entitled TISSUE CROSSLINKING FOR TREATMENT OF SNORING AND OBSTRUCTIVE SLEEP APNEA, now U.S. Pat. No. 9,192,507; U.S. patent application Ser. No. 14/869,420, filed Sep. 29, 2015, entitled TISSUE CROSSLINKING FOR TREATMENT OF SNORING AND OBSTRUCTIVE SLEEP APNEA, now U.S. Pat. No. 9,918,870; and U.S. patent application Ser. No. 14/685,539, filed Apr. 13, 2015, entitled CROSSLINKER ENHANCED REPAIR OF KNEE MENISCUS, now U.S. Pat. No. 9,492,592. The above-referenced patent applications are hereby incorporated by reference in their entirety into the present application.

### BACKGROUND

### 1. Field

Embodiments of the invention relate to methods for treating collagenous tissue. More specifically, embodiments of the invention relate to methods for non-toxic crosslinking reagent treatment of thermally shrunken collagenous tissue to immediately improve mechanical properties of the tissue.

### 2. Related Art

Connective tissue overuse injuries, such as overuse of tendons and/or ligaments, are common and are caused by repetitive strain that leads to the formation of micro-tears within the connective tissue. Accumulation of micro-tears progressively weakens collagen within the connective tissue. Similarly, joint injuries often involve damage and loosening of supporting ligaments and/or tendons and/or joint capsules. These injuries decrease tissue integrity and increase joint laxity. Joint laxity may cause deterioration of the joint over time and lead to osteoarthritis in the joint. Laxity of connective tissue (e.g., tendons, ligaments, joint capsules) can disrupt neuromuscular function by reducing passive motion constraint and thereby affecting muscle coactivation, which is crucial for maintaining joint stability.

Clinically, connective tissue injuries are linked to persistent pain, joint instability, and a higher likelihood of recurrent injuries, impacting the patient's quality of life. For example, conditions such as shoulder instability, Achilles tendinopathy, rotator cuff tears, shoulder bursitis, patellar tendinopathy, lateral epicondylitis (tennis elbow), medial epicondylitis (golfer's elbow), finger flexor tendons, ulnar collateral ligament (UCL) tears, biceps tendinopathy, medial collateral knee ligament laxity, anterior cruciate ligament (ACL) tears, posterior tibial tendinopathy, sacroiliac ligament tear, glenohumeral ligament tears, and shoulder joint capsule laxity often involve the elongation and degeneration of connective tissue fibers due to injury, chronic overuse and/or micro-tear progression. There is need for a minimally invasive method to reduce collagenous tissue (e.g., connective tissue) laxity while maintaining or immediately restoring the strength and other elastic-plastic and viscoelastic properties of the collagen within the tissue, and arrest the progression of tissue degradation, thereby enhancing the durability and function of collagenous tissue, such as connective tissue (e.g., tendons, ligaments, joint capsules).

As an example, shoulder instability, associated with shoulder pain, humeral head dislocations and subluxations, and associated disability can result from traumatic injury and accumulated tissue degradation due to overuse such as in sports and activities requiring repetitive overhead motions. Conventional treatments involve physical therapy, rest, pain medications, and surgical capsulorrhaphy. Capsulorrhaphy involves tightening the capsule and ligaments around the shoulder joint and can be done using arthroscopic or open procedures. There is need for a minimally invasive method to reduce laxity in shoulder capsular and ligamentous tissues while maintaining or immediately restoring the strength and other elastic-plastic and viscoelastic properties of the collagen in these tissues.

Snoring is a common condition that can be associated with a more serious sleep related breathing disorder called obstructive sleep apnea (OSA) where breathing is temporarily interrupted by obstruction of the airways resulting in episodes of hypoxia and disrupted sleep. The etiologies of snoring and OSA are not fully understood but both are related to obstruction of air flow through air passages, either partially in the case of snoring or completely in the case of OSA. Typical treatments for snoring and OSA include surgery to modify the geometry of the airways or oral appliances which either temporarily adjust the airway geometry by holding the jaw in a certain position or they apply continuous positive air pressure (CPAP) to keep airways open. There is a need for a treatment that can both shrink and mechanically reinforce the soft palate tissue to reduce the symptoms and incidence of snoring and OSA.

Urinary stress incontinence is a condition where movement or activity (such as coughing, laughing, sneezing, running, etc) causes involuntary leakage of urine. Treatment typically begins with behavioral changes, rehabilitation to strengthen pelvic floor muscles that promote urine storage, or pharmacologic therapy. If the previous methods are unsuccessful there are surgical treatments to create slings that support the urethra tube, bladder, or retropubic periosteum. A recent technique uses radiofrequency (RF) energy to shrink the endopelvic fascia and provide support for the urethra and bladder neck. There is a need for a minimally invasive therapy that can tighten the endopelvic fascia and maintain or immediately restore the strength and other elastic-plastic and viscoelastic properties of the tissue to support the bladder and nearby tissues to treat urinary stress incontinence.

Arthroscopic and minimally invasive methods such as laser and radiofrequency energy delivery have been employed to selectively shrink and tighten degraded tissue. These thermal shrinkage techniques aim to restore stability to joints and reduce the laxity of over-stretched tissues. RF or laser thermal treatment works by altering the three-dimensional, triple-helical architecture of the collagen fibers, from the ordered, cross-linked (crystalline) arrangement to a denatured contracted random coil. As collagen is heated to a critical temperature, heat-liable intramolecular hydrogen bonds within the tissue break, and protein in the tissue undergoes a phase transition from a highly ordered crystalline structure to a random-coil state, similar to melting. Typically, this “denaturation” occurs at approximately 55-70° C., the “shrinkage temperature,” although the temperature varies between tissues and species and is higher with increased collagen, especially type I collagen, and increased tensile load on the tissue.

However, thermal shrinkage techniques have proven largely ineffective for load carrying, highly stressed tissues over the long term, as the treated tissues have diminished tensile strength properties and often rapidly stretch back, failing to provide lasting stability. The thermally treated tissue is mechanically weaker due to the disruption of the hydrogen bonds and polypeptide chains to achieve the tissue shrinkage. Thus, the mechanical properties of tissue deteriorate with increased application of thermal energy for thermal shrinkage of the tissue. Even limited delivery of thermal energy may reduce elastic-plastic and viscoelastic mechanical characteristics of the already degraded tissue. The degradation of mechanical properties of the already-degraded tissue lessens treatment durability and/or prolonged functional improvement of the involved joints. Healing and regeneration of these shrunk tissues is sometimes possible. However, it requires long periods of load avoidance on the tissue and immobilization of the associated joint. Typically, adequate restoration of shrunk tissue mechanical properties is not achieved even after long periods (weeks) of load avoidance and joint immobilization. Additionally, the amount and consistency of shrinkage are difficult to measure and control in situ, further complicating the use of thermal energy to shrink native tissues in situ.

Thus, while the application of thermal energy to degraded tissue treats the issue of tissue laxity in the short term, there exists a need for increasing the durability of such thermally shrunk tissue to prolong the shrinkage results and for immediate restoration of the mechanical properties of the tissue otherwise affected by the thermal tissue shrinkage procedure.

### SUMMARY

The present disclosure introduces the use of non-toxic protein crosslinkers (e.g., genipin) for application before, during, and/or after thermal tissue shrinkage to immediately improve and/or restore material properties (e.g., mechanical properties) of the tissue otherwise affected by the thermal energy and maintain the shrinkage of the tissue. Examples of how delivery of non-toxic crosslinkers to collagenous tissue improves material properties and mechanical integrity are described in U.S. Pat. Nos. 9,084,772; 9,101,602; 7,435,722; 8,153,600; 8,022,101; 8,211,938; 10,278,947; 10,980,771; 8,450,276; 8,119,599; 8,198,248; 8,283,322; 9,192,507; 9,918,870; and 9,492,592, which are incorporated by reference in their entirety. Genipin is a protein crosslinker and, in an aqueous solution, self-polymerizes and can form a mesh of load-carrying oligomers that distribute throughout a collagenous tissue and covalently bond to available collagen amines. Genipin polymeric mesh has demonstrated the capability to immediately enhance the mechanical properties of various load-carrying collagenous tissues that have not been heat denatured. Therefore, the current disclosure relates to a minimally invasive procedure to contact collagenous tissue with genipin in a buffered carrier before, during, and/or after thermal tissue shrinkage to maintain or immediately improve material properties of the load-carrying collagenous tissue that would otherwise degrade due to the thermal tissue shrinkage procedure. The disclosed methods, systems (e.g., kits), and/or devices for delivering a protein crosslinker before, during, and/or after tissue shrinkage immediately provide sufficient restoration/improvement of material properties to increase and prolong the benefits of thermal shrinkage of collagenous tissues, making thermal shrinkage a more viable and durable treatment option for laxity in load carrying collagenous tissue, such as connective tissue (e.g., ligaments, tendons, joint capsules). For purposes of the present disclosure, “immediately” at least means a restoration/improvement of mechanical properties within twenty-four hours of treatment of the target tissue.

Embodiments of the present disclosure immediately restore and/or improve mechanical integrity and material properties of collagenous tissues, such as connective tissue (e.g., ligaments, tendons, joint capsules), shrunken via thermal energy.

In some embodiments, the techniques described herein relate to a method of restoring and/or improving the mechanical integrity and restoring and/or improving the material properties of load-carrying tissues, such as tendons or ligament, that have been or will be shrunk by applying thermal energy.

In some embodiments, the thermal energy is applied and the genipin is delivered to the degraded target tissue through a minimally invasive procedure, such as an arthroscopic technique. In some embodiments, the procedure may comprise applying thermal energy along the surface of the target degraded tissue to thereby shrink the target degraded tissue. The genipin may be solubilized in a buffered aqueous liquid to form a genipin reagent and delivered via injection of the reagent into the degraded tissue.

In some embodiments, the genipin reagent may be injected and/or topically applied simultaneously with the application of thermal energy to the target tissue. The genipin reagent may be injected into the target tissue as thermal energy is applied to the target tissue. In some embodiments, the genipin may be topically applied to the target tissue as thermal energy is applied. In some embodiments, the genipin may be injected and/or topically applied after the application of thermal energy to shrink the target tissue. In some embodiments, the genipin may be injected and/or topically applied prior to the application of thermal energy to shrink the target tissue.

In some embodiments, the genipin may be delivered to the target tissue after the tissue has been shrunken via thermal energy. The thermal energy may be applied to the target tissue through an incision made in the target tissue. The genipin may be added to a degradable coating that contains a plasticizer, and the coating may be applied to a suture. The suture may then close the incision and contact the target pre-shrunk tissue, such that the genipin is delivered to the pre-shrunk tissue via release from the degradable coating on the suture.

In some embodiments, the techniques described herein relate to a surgical system (e.g., kit), further including a device or devices to apply thermal energy to the target degraded tissue to shrink the tissue and a device or devices or surgical kit to deliver the genipin and/or genipin reagent to the tissue before, during, and/or after thermal energy application.

The thermal energy may be applied to the target tissue using a thermal energy device via a minimally invasive technique to provide contact between the thermal energy device and the lax tissue. The thermal energy device may comprise a hollow through which the genipin or genipin reagent may be delivered to the target tissue. The genipin or genipin reagent may travel through the hollow and onto or into the target tissue before, during, and/or after thermal shrinkage of the tissue. Thus, a single device may both apply thermal energy to shrink the tissue and deliver the genipin or genipin reagent into the target tissue, without the need to remove the thermal energy device or insert a separate delivery device into the target tissue.

In some aspects, the techniques described herein relate to a method of delivering a protein crosslinker to a target tissue at a treatment site. For example, the method may include creating a minimally invasive incision at the treatment site, then inserting a thermal energy device into the minimally invasive incision to thereby access the target tissue. In some aspects, the method may further include applying thermal energy to the target tissue using the thermal energy device. Applying thermal energy further may include contacting at least a portion of the target tissue with the thermal energy device to thereby shrink the target tissue. In some aspects, the method may further include contacting the shrunk target tissue with a protein crosslinker to thereby restore the mechanical properties of the shrunk target tissue.

In some aspects, contacting at least a portion of the shrunk target tissue with the protein crosslinker includes injecting the protein crosslinker into the shrunk target tissue.

In some aspects, the protein crosslinker is genipin and the genipin is solubilized in a buffer to form a genipin reagent, The concentration of genipin may be within a range of 10 mM to 100 mM.

In some aspects, the techniques described herein include setting a predetermined amount of time for which the thermal energy device contacts the portion of the target tissue.

In some aspects, the thermal energy device defines a proximal end, a distal end, and a hollow extending from the distal end to the proximal end. In some aspects, contacting the shrunk target tissue with the protein crosslinker includes injecting the protein crosslinker through the hollow.

In some aspects, contacting the shrunk target tissue with the protein crosslinker includes placing at least one of a microneedle patch, a biodegradable film, a biodegradable gel, or a protein crosslinker-coated suture on the shrunk target tissue.

In some aspects, the thermal energy device includes a bipolar radiofrequency probe having at least one active electrode and at least one return electrode.

In some aspects, the techniques described herein relate to a dual applicator for treating a target tissue. The dual applicator may comprise a housing, a thermal energy probe received in the housing, an energy supply connection interface, and an injection needle. The thermal energy probe may be longitudinally translatable within the housing. The energy supply connection interface may be coupled to the thermal energy probe and configured to supply thermal energy to the thermal energy probe. The injection needle may be included in the housing and coupled to a trigger. Actuating the trigger may cause longitudinal translation of the injection needle to thereby adjust the depth of the injection needle inserted into the target tissue.

In some aspects, the dual applicator may further include a Luer lock connector operable to receive a syringe vial.

In some aspects, the dual applicator may further including a depth gauge, wherein the depth gauge indicates a position of the injection needle.

In some aspects, the dual applicator may further comprise a depth stop that may limit the position the injection needle when extended into the target tissue.

In some aspects, actuating the energy supply connection interface may longitudinally translate the thermal energy probe.

In some aspects, the dual applicator may further include an aperture, wherein the aperture provides a view of the syringe vial connected to the Luer lock connector.

In some aspects, the thermal energy probe of the dual applicator is a bipolar radiofrequency probe comprising at least one active electrode and at least one return electrode.

In some aspects, the injection needle of the dual applicator is received through a hollow in the housing.

In some aspects, the techniques described herein relate to a method of treating a target tissue. The method may include creating a minimally invasive incision, and inserting, through the minimally invasive incision, a distal end of a dual applicator. In some aspects, the dual applicator includes a thermal energy probe, an injection needle; a trigger, and a vial containing a protein crosslinker. In some aspects, the method may include applying thermal energy to the first region via the thermal energy probe, then adjusting a position of the injection needle relative to the target tissue. The adjustment may include contacting a first region of the target tissue with the thermal energy probe. In some aspects, the method may include applying thermal energy to the first region via the thermal energy probe, then adjusting a position of the injection needle relative to the target tissue. The adjustment of the injection needle may include inserting the injection needle into the first region. The method may further include actuating the trigger to translate the injection needle within the dual applicator, and delivering the protein crosslinker to the first region from the vial.

In some aspects, the method may further include performing a second adjustment of the position of the injection needle, wherein the second adjustment of the position of the injection needle includes inserting the injection needle into a second region by actuating the trigger to translate the injection needle out of the first region and into the second region. In some aspect, the method further includes delivering the protein crosslinker to the second region from the vial.

In some aspects, applying thermal energy to the first region comprises applying a voltage within a range of 10 volts to 500 volts to the first region.

In some aspects, applying thermal energy to the first region includes applying thermal energy to the first region until the first region reaches a predefined temperature.

In some aspects, applying thermal energy to the first region further includes applying thermal energy to the first region of the target tissue until the target tissue has shrunk by within a range of 10% to 50% of a pre-treatment length of the target tissue.

In some aspects, the dual applicator further includes an energy supply connection interface coupled to the thermal energy probe and configured to supply thermal energy to the thermal energy probe, wherein adjusting the position of the thermal energy probe further includes moving the energy supply connection interface to thereby translate the thermal energy probe.

This summary is provided to introduce a selection of concepts in a simplified form that are further described below in the detailed description. This summary is not intended to identify key features or essential features of the claimed subject matter, nor is it intended to be used to limit the scope of the claimed subject matter. Other aspects and advantages of the invention will be apparent from the following detailed description of the embodiments.

### BRIEF DESCRIPTION OF FIGURES

Embodiments of the present disclosure are described in detail below with reference to the attached drawing figures, wherein:

FIG. 1 illustrates a method of delivering a protein cross-linker to target tissue subjected to thermal shrinkage;

FIG. 2A illustrates a dual applicator for treating a target tissue for some embodiments;

FIG. 2B illustrates a perspective view of the dual applicator of FIG. 2A;

FIG. 3 illustrates a second embodiment of a dual applicator for treating a target tissue for some embodiments;

FIG. 4A illustrates a third embodiment of a dual applicator for treating a target tissue for some embodiments;

FIG. 4B illustrates a perspective view of the dual applicator of FIG. 4A;

FIG. 5A illustrates a fourth embodiment of a dual applicator for treating a target tissue for some embodiments;

FIG. 5B illustrates a perspective view of the dual applicator of FIG. 5A;

FIG. 6 illustrates an exemplary method of treating a target tissue with a dual applicator; and

FIG. 7 illustrates an exemplarily system for treating a target tissue.

### DETAILED DESCRIPTION

The following detailed description references the accompanying drawings that illustrate specific embodiments in which the present disclosure can be practiced. The embodiments are intended to describe aspects of the present disclosure in sufficient detail to enable those skilled in the art to practice the present disclosure. Other embodiments can be utilized and changes can be made without departing from the scope of the present disclosure. The following detailed description is, therefore, not to be taken in a limiting sense. The scope of the present disclosure is defined only by the appended claims, along with the full scope of equivalents to which such claims are entitled.

In this description, references to “one embodiment,” “an embodiment,” or “embodiments” mean that the feature or features being referred to are included in at least one embodiment of the technology. Separate references to “one embodiment,” “an embodiment,” or “embodiments” in this description do not necessarily refer to the same embodiment and are also not mutually exclusive unless so stated and/or except as will be readily apparent to those skilled in the art from the description. For example, a feature, structure, act, etc. described in one embodiment may also be included in other embodiments, but is not necessarily included. Thus, the technology can include a variety of combinations and/or integrations of the embodiments described herein.

Embodiments of the present disclosure are generally directed to systems (e.g., kits), devices, and methods for immediately restoring and/or improving the mechanical integrity and material properties (e.g., mechanical properties) of collagenous tissue before, during, and/or after thermally shrinking the tissue. For purposes of the present disclosure, “immediately” at least means a restoration/improvement of mechanical properties within twenty-four hours of treatment of the target tissue. Collagenous tissue, such as connective tissues (e.g., ligaments, tendons, or joint capsules), may become lax due to injury, overuse and/or repetitive strain that damages the collagen matrix. Applying thermal energy shrinks the collagenous degraded tissues to decrease laxity. However, the material properties and microstructure of collagenous tissues are affected by thermal energy applied during thermal shrinkage, due to the disruption of hydrogen bonds and polypeptide chains within the degraded target tissue. Healing and regeneration of these shrunk tissues is generally inadequate and requires long periods (weeks) of load avoidance on the tissue and immobilization of the associated joint while the limited healing is occurring. Previously, the capability of protein crosslinkers (e.g., genipin) to immediately and adequately restore the elastic-plastic and viscoelastic mechanical properties of heat denatured and disrupted collagenous tissues has not been known, investigated or demonstrated. Indeed, traditional work using protein crosslinkers to restore mechanical properties of collagenous tissues does not involve thermal shrinkage and relies on the ability of a protein crosslinker to form crosslinks within ordered collagen networks. As such, though genipin has previously been shown to enhance the mechanical properties of collagenous tissues, its ability to restore such mechanical properties to collagenous tissues within collagen matrices disrupted by heat (e.g., after thermal shrinkage) was previously unknown. Therefore, the present disclosure describes systems (e.g., kits), methods, and/or devices for applying non-cytotoxic protein crosslinkers (e.g., genipin) before, during, and/or after thermal shrinkage to immediately restore and/or improve material properties of degraded and heat-disrupted collagenous tissues, thereby improving the mechanical integrity of the shrunken tissue. In some aspects, applying a protein crosslinker before, during, and/or after thermal shrinkage may improve the mechanical integrity of the shrunken tissue by improving material properties that may include, for example, mechanical properties. Mechanical properties include at least one of strength, fatigue resistance, viscoelastic and elastic-plastic characteristics such as stress-relaxation, creep, Young's modulus, yield stress, yield strain, ultimate tensile stress, ultimate tensile strain, resilience, toughness, load-history dependence, hysteresis, and storage and loss moduli.

In some embodiments, connective tissues other than load-carrying ligaments, tendons, or joint capsules could become lax due to injury, overuse, and/or repetitive strain that damages the collagen matrix. Collagenous tissues such as endopelvic fascia or the soft palate could have thermal energy applied to shrink the collagen to remove laxity causing loss of tissue constraint or obstructions (such as in obstructive sleep apnea involving a flaccid soft palate). For example, non-ablative levels of RF energy and non-toxic protein crosslinkers may be applied to the endopelvic fascia in a minimally invasive approach to shrink and stabilize the tissue to treat urinary stress incontinence. Thermal energy may be applied before or after the addition of a non-cytotoxic protein crosslinker. In some embodiments, the non-cytotoxic protein crosslinker is applied after RF energy shrinkage of the tissue to immediately restore and/or improve the material properties of the shrunken/degraded tissue.

In some embodiments, thermal energy and non-toxic protein crosslinkers are applied to the connective and muscle tissue of the soft palate. For example, RF energy and a protein crosslinker may be applied to the soft palate to shrink and tighten the tissue and to immediately improve the elastic-plastic and viscoelastic mechanical properties of the tissue to reduce the incidence and effects of snoring and OSA. The reduction in tissue laxity may be mechanically reinforced and preserved with the application of a non-cytotoxic protein crosslinker such as genipin. An anti-inflammatory drug may be given prophylactically, periprocedurally, or postoperatively to the RF and crosslinker treated tissues due to the vascular nature of the soft palate tissue.

In some embodiments, the thermal energy may be applied to grafting material such as autografts or allografts after the initial implantation to repair an injury. For example, RF thermal energy may be used to revise an autograft used to repair an ACL after the initial procedure if the implant begins to fail due to laxity in the graft or intervening creep lengthening of the graft. Following the RF thermal energy shrinking of the graft material, a non-cytotoxic protein crosslinker may be applied to immediately mechanically reinforce the autograft that has been shrunk to decrease and/or remove laxity.

In some embodiments, thermal energy is applied to at least a portion of collagenous tissue at the target treatment site using a thermal energy device. For example, thermal energy may be applied to at least a portion of a connective tissue, such as a ligament, tendon, or joint capsule at the target treatment site using a thermal energy device. In some embodiments, thermal energy may be applied to at least a portion of tissue other than connective tissue, such as the soft palate or graft materials. In some embodiment, the target tissue may be degraded prior to thermal energy application. In some embodiments, radio frequency (RF) energy may be applied to the target tissue using a thermal energy device. A variety of types of thermal energy may be used, including, but not limited to, thermal energy produced by radiofrequency, ultrasound, lasers, broadband light devices, infrared light devices, and optical energy devices. The thermal energy device may comprise a proximal end and a distal end. The thermal energy device may comprise a hollow through which a protein crosslinker may be injected into the target tissue therethrough, as discussed further herein. Exemplary thermal energy devices may include bipolar RF probes, monopolar RF probes, laser devices, broadband light devices, infrared light devices, and optical energy devices.

In some embodiments, the delivery device may be a dual applicator for applying both thermal energy and the protein crosslinker to the target tissue, as further discussed herein.

In some embodiments, the distal end of the thermal energy device is inserted through a minimally invasive incision to contact the target tissue, then thermal energy is applied along the surface of the target tissue such as with sweeps across the tissue surface using a bipolar or monopolar RF probe. In some embodiments, a minimally invasive incision is made to access the target tissue, and a second incision is made in the target tissue before or while the distal end of the thermal energy device is contacting the target tissue such that thermal energy can be applied to the interior of the target tissue. In some embodiments, thermal energy is applied at the surface of the skin (non-invasively) and the protein crosslinker (genipin) is applied to the target tissue by an injection of the crosslinker in a carrier liquid (e.g., buffer).

In some embodiments, a protein crosslinker, such as genipin, is delivered to the target tissue following the application of thermal energy to the target tissue to shrink the tissue. For example, the protein crosslinker may be injected into the target tissue after suturing a minimally invasive incision used for the thermal shrinkage procedure. In some embodiments, a protein crosslinker, such as genipin, is delivered to the target tissue during the application of thermal energy to the target tissue. In some embodiments, the protein crosslinker (e.g., genipin) may be delivered to the target tissue at various times during the procedure. For example, a protein crosslinker, such as genipin, may be delivered both before and after the application of thermal energy to the target tissue. In some embodiments, a protein crosslinker, such as genipin, may be delivered both during and after the application of thermal energy. In some embodiments, a protein crosslinker, such as genipin, may be delivered before, during, and after the application of thermal energy to the target tissue.

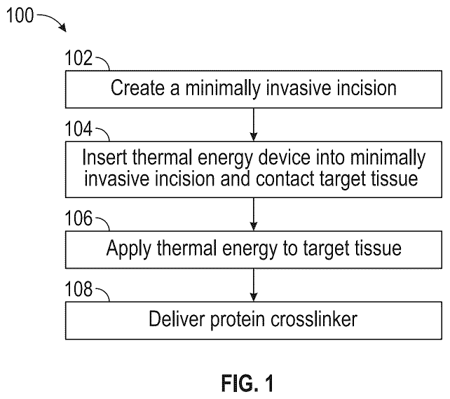

Referring now to FIG. 1, a method 100 of delivering a protein cross-linker to target tissue subjected to thermal shrinkage is depicted. Step 102 may comprise making a minimally invasive incision to access a target tissue. Step 104 may comprise inserting a thermal energy device into the incision and adjacent the target tissue. In some embodiments, the target tissue may be degraded due to microtears, overuse, or repetitive strain, causing the tissue to have increased laxity. Step 106 may comprise applying thermal energy to the target tissue. The method 100 may further comprise step 108 delivering a protein crosslinker to the target tissue. Delivering the protein crosslinker to a heat-shrunk target tissue may improve the mechanical properties of the target tissue.

In some embodiments, step 104 may include making a second incision in the target tissue and inserting a thermal energy device into the second incision to reach the interior of the tissue. In some embodiments, step 108 may comprise delivering a protein crosslinker to the target tissue via an injection of liquid protein crosslinker, application of a microneedle patch, protein crosslinker coated suture, a biodegradable film, a biodegradable gel, or any combination thereof.

In some embodiments, the thermal energy device may be a bipolar RF probe or a monopolar RF probe. The bipolar RF probe may comprise a local active electrode and a local return electrode. The use of both a local active electrode and local return electrode applies the electric current to the target tissue through a controlled path. In some embodiments, the bipolar RF probe may comprise an array of active electrodes and an array of return electrodes. In some embodiments, the bipolar RF probe may comprise a single active electrode and an array of return electrodes. In some embodiments the bipolar RF probe may comprise an array of active electrodes and a single return electrode. In some embodiments, the thermal energy device may be a dual applicator device for applying thermal energy to the tissue and applying a protein-crosslinker (e.g., genipin), as discussed further herein.

In some embodiments, upon insertion of a thermal energy device (step 104), a power source may be used to provide electrical current to the thermal energy device to thereby apply thermal energy to the target tissue (step 106). Such current may flow through the thermal energy device and into the target tissue, thereby heating the tissue directly. Additionally, the electrical current passing through the thermal energy device may heat an electrically conductive fluid present in the area surrounding the target tissue, causing the conductive fluid to indirectly heat the target tissue. In some embodiments, both the direct and indirect heating of the target issue may cause thermal shrinkage of the target tissue. The thermal energy device may be connected to the power source through a single wire or multiple wires. The thermal energy device may be configured to apply electrical current (both directly and indirectly) to heat the target tissue until the tissue reaches a predefined temperature. For example, the predefined target temperature may be within a range of 59° C. to 70° C., such as between 60° C. and 65° C. It is to be understood that these temperature ranges may be modified or altered based upon the specific conditions of the target tissue to be treated and based on the desired shrinkage amount. The power source may heat the thermal energy device via an electrical current until the thermal energy device reaches the target temperature.

In some embodiments, during step 106, the thermal energy device may be configured to maintain the target temperature while the thermal energy device contacts a surface or the interior of the target tissue. The predefined temperature may be set based on the amount of shrinkage desired. In some embodiments, the target temperature may be reached by modifying a voltage applied to the thermal energy device by the power source. An applied voltage may be modified by an operator of the power source. The energy delivered may be controlled by a pre-programmed computer controller with adjustable inputs and switches such as a foot-operated switch or by a digital interface.

In some embodiments, the thermal energy device is a radiofrequency (RF) bipolar electrode connected to the power source. The operator may select a particular voltage level of the power source. In some embodiments, the power source is configured to output a RF voltage level within the range of 10 volts to 500 volts. In some embodiments, the power source is configured such that the operator can specify the frequency at which the voltage is applied. As such, the power source may be configured to apply voltage at a frequency within a range of 100 kHz and 700 kHz. In some embodiments, the operator may choose the applied voltage and frequency based on the state of the target tissue and the dimensions of the target tissue. For example, the operator may choose the applied voltage and frequency based on the level of degradation of the target tissue and the size of the target tissue to be shrunk. In some embodiments, the operator may choose the voltage and frequency to achieve a certain temperature at the target tissue site.

In some embodiments, step 106 may further include continuing to apply heat via the thermal energy probe at the predefined temperature to the target tissue for a predefined set amount of time. The amount of time may be set based on the amount of shrinkage desired. Once the predefined temperature has been reached, the thermal energy device may be configured to maintain the predefined temperature while the thermal energy device is moved along a surface of or within the target tissue. In some embodiments, the thermal energy device is applied to the target tissue until the target tissue is shrunk by a range of 10% to 50% of the pre-treatment length of the target tissue. It is to be understood that the shrinkage amount may be altered depending on the initial state of the target tissue, such as how degraded the target tissue is prior to the procedure.

In some embodiments, step 106 may further comprise moderating the thermal energy applied to the target tissue by application of a cryogenic agent to the heated target tissue. For example, step 106 may comprise applying a cryogen spray (1,1,1,2-tetrafluoroethane) or liquid nitrogen to the target tissue. The cryogenic agent may be applied to the heated target tissue such that the target tissue does not reach a predefined upper temperature threshold.

In some embodiments, steps 106 and 108 may be performed simultaneously. For example, steps 106 comprise inserting the thermal energy device (e.g., bipolar RF probe) through a minimally invasive incision to contact a target tissue and applying thermal energy to shrink the target tissue. During tissue shrinkage (step 106), a protein crosslinker may be simultaneously delivered to the target tissue (step 108), via injection and/or topical application through a hollow of the thermal energy device. The thermal energy device may deliver the genipin solution prior to, during, and/or after thermal shrinkage of a target tissue.

In some embodiments, step 108 may comprise delivering the protein crosslinker to the target tissue repeatedly at various times to maintain the improved material properties and mechanical integrity provided by the protein crosslinker application before, during, and/or after thermal shrinkage. The number of times the protein crosslinker (e.g., genipin) is repeatedly administered may be based on the needs of each individual patient. For example, a patient may undergo thermal shrinking of target collagenous tissue (e.g., connective tissue, such as tendon, ligament, or joint capsule) treated with a genipin reagent at or around the time of the thermal shrinking procedure. In some embodiments, step 108 may further comprise applying a protein crosslinker, such as genipin, to the target tissue after a certain amount of time has passed since the thermal shrinking (e.g., one month, six months). The reapplication of genipin need not require an incision to access the target tissue, depending on the local anatomy, and may be applied through a simple injection or ultrasound or fluoroscopy guided injection. If access to the target tissue cannot be achieved without an incision, the protein crosslinker (e.g., genipin) may be re-administered through a minimally invasive incision.

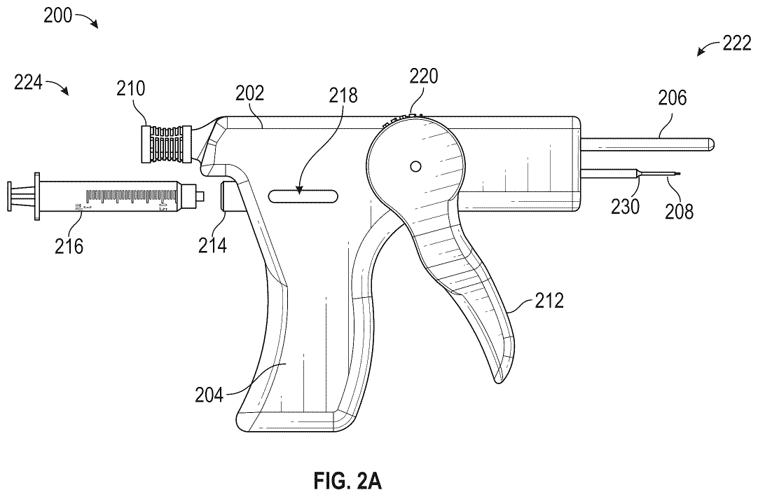

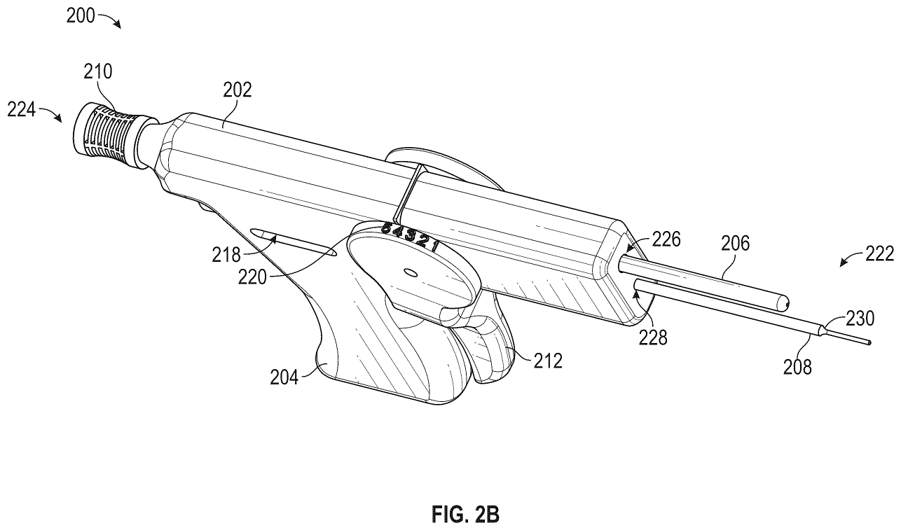

FIGS. 2A-B illustrate an exemplary dual applicator 200 which may be used to perform the method of applying thermal energy to the tissue and applying a protein cross-linker to the heated tissue, such as method 100. The dual applicator 200 may comprise a housing 202 with a handle 204. Housing 202 may further comprise a thermal energy probe 206, an injection needle 208, an energy supply connection interface 210 and a trigger 212. In some embodiments, the dual applicator 200 may also comprise a connector 214 onto which a syringe vial 216 may be connected, and an aperture 218 through which the syringe vial 216 may be viewed when connected. Dual applicator 200 may further comprise a depth gauge 220 positioned on trigger 212, as shown in FIG. 2B.

In some embodiments, dual applicator 200 may be used to treat a target tissue. For example, a minimally invasive incision may be made and a distal end 222 of dual applicator 200 may be inserted through the minimally invasive incision such that thermal energy probe 206 may contact the target tissue. Injection needle 208 may be received in housing 202 and used to inject a protein crosslinker on or into the target tissue. For example, when distal end 222 is inserted through the minimally invasive incision, injection needle 208 may be used to inject protein crosslinker into the target tissue.

In some embodiments, thermal energy probe 206 may be a bipolar RF probe, a monopolar RF probe, a laser device, a broadband light device, an infrared light device, or an optical energy device. In an exemplary embodiment, thermal energy probe 206 may be a RF probe. When thermal energy probe 206 is an RF probe, the RF probe may comprise a metal electrode, such as a platinum electrode, a gold electrode, a stainless steel electrode, a tungsten electrode, or any combination thereof. In some embodiments, when thermal energy probe 206 is an RF probe, the RF probe may further comprise an insulating shaft, such as a polyether ketone shaft, a Teflon shaft, a silicone shard, a polyimide shaft, a polyurethane shaft, or any combination thereof. In some embodiments, the RF probe may further comprise a temperature sensor such as a thermocouple, a thermistor, or both a thermocouple or thermistor. Further, thermal energy probe 206 may be one of a monopolar RF probe or a bipolar RF probe. As described above, the bipolar RF probe may comprise at least one local active electrode and at least one local return electrode, which allows applied electric current to travel through the target tissue in a controlled path.

In some embodiments, energy supply connection interface 210 may be coupled to thermal energy probe 206 such that thermal energy probe 206 may be connected to an energy/power supply. In some embodiments, energy supply connection interface 210 is a metallic threaded connector. In some embodiments, energy supply connection interface 210 is a standard plug. In some embodiments, energy supply connection interface 210 may route power generated by the energy/power supply to the thermal energy probe 206 such that thermal energy may be generated at thermal energy probe 206. In some embodiments, energy/power routed to the thermal energy probe 206 may be regulated by a pre-programmed computer controller with adjustable inputs and switches such as a foot-operated switch or by a digital interface.

In some embodiments, energy supply connection interface 210 may be used to control a position of thermal energy probe 206. Housing 202 may comprise a hollow 226 (as shown in FIG. 2B) in which thermal energy probe 206 is positioned, such that the thermal energy probe 206 extends between proximal end 224 and distal end 222 through the hollow, thereby extending through the length of housing 202. The thermal energy probe 206 may be movable within hollow 226 by manipulating the energy supply connection interface 210 to thereby adjust a position of the thermal energy probe 206. For example, the thermal energy probe 206 may be longitudinally translatable through housing 202. For example, thermal energy probe 206 may be directly connected to energy supply connection interface 210 at a proximal end 224 of dual applicator 200 and pulling energy supply connection interface 210 may cause thermal energy probe 206 to translate toward proximal end 224. Similarly, pushing on energy supply connection interface 210 may cause thermal energy probe 206 to translate toward distal end 222. As such, thermal energy probe 206 may be longitudinally translatable through housing 202. Thus, when distal end 222 is inserted through a minimally invasive incision, the depth of thermal energy probe 206 in the minimally invasive incision may be adjusted by longitudinally translating thermal energy probe 206 via manipulation of the energy supply connection interface 210.

In some embodiments, injection needle 208 may be positioned in a hollow 228 in housing 202 (as shown in FIG. 2B), extending between proximal end 224 and distal end 222. In some embodiments, injection needle 208 may be used to inject a protein crosslinker on or into a target tissue. In some embodiments, syringe vial 216 may house the protein crosslinker. Syringe vial 216 may connect to connector 214 such that the protein crosslinker in syringe vial 216 may be injected into injection needle 208, and thereby into the target tissue.

In some embodiments, syringe vial 216 may be a standard syringe that connects to connector 214 via a Luer lock or a Luer slip. In some embodiments, syringe vial 216 may comprise a stepped button such that a pre-set controlled volume of the protein crosslinker may be delivered to injection needle 208 with each press of the stepped button. A volume of protein crosslinker in syringe vial 216 may be viewed through aperture 218 such that a user of dual applicator 200 may view how much protein crosslinker is left in syringe vial 216 and how much protein crosslinker has been administered.

In some embodiments, injection needle 208 may be coupled to trigger 212 such that a position of injection needle 208 may be controlled by trigger 212. Actuating trigger 12 may cause adjustment of injection needle 208 position. In some embodiments, actuating trigger 12 may cause longitudinal translation of injection needle 208. For example, pressing trigger 212 may cause injection needle 208 to translate toward distal end 222, while releasing trigger 212 may cause needle 208 to translate toward proximal end 224. In some embodiments, pulling trigger 212 toward proximal end 224 may cause injection needle 208 to translate toward distal end 222. Similarly, pushing trigger 212 toward distal end 222 may cause injection needle 208 to translate toward proximal end 224. The adjustment of the injection needle 208 position may allow a user to change the depth at which the protein crosslinker is injected into the target tissue.

A user of dual applicator 200 may view the position of injection needle 208 using depth gauge 220. For example, as shown in FIG. 2B, depth gauge 220 may comprise a range of values indicating the extent to which injection needle 208 extends. When a user presses/releases trigger 212 to a certain value within the range, the value may indicate the depth the needle 208 is injected. For example, when trigger 212 is pulled or pressed to number “5” on depth gauge 220, injection needle 208 may be fully extended. Similarly, when trigger 212 is released or pushed to number 1 on depth gauge 220, injection needle 208 may be fully retracted. In some embodiments, depth gauge 220 is a range from 1-5 mm, with “5” referring to a depth of 5 mm and “1” referring to a depth of 1 mm. As such, when distal end 222 is inserted through a minimally invasive incision, the depth of injection needle 208 in the minimally invasive incision may be adjusted by pushing/pulling or pressing/releasing trigger 212. Such a feature is advantageous because after a volume of protein crosslinker is injected through injection needle 208 into the target tissue, injection needle 208 may be pulled out of the target tissue and distal end 222 may be positioned at a new location without having to remove dual applicator 200 from the minimally invasive incision. In this way, multiple injections of protein crosslinker may be administered to various areas of a target tissue without removing the distal end 222 of dual applicator 200 from the minimally invasive incision. A depth stop 230 may limit the position of injection needle 208 when extended into the target tissue.

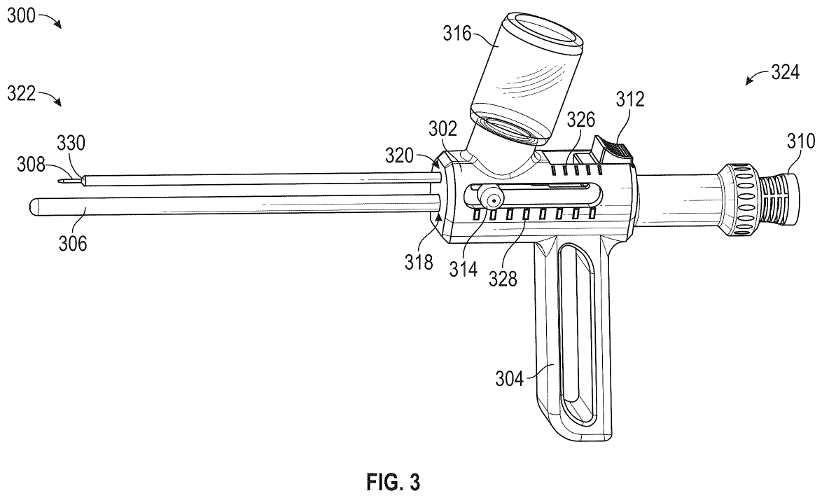

FIG. 3 illustrates a second embodiment of the dual applicator 300 which may be used to perform the method of applying thermal energy to the tissue and applying a protein cross-linker to the heated tissue, such as method 100. The dual applicator 300 may comprise a housing 302, a handle 304, a thermal energy probe 306, an injection needle 308, an energy supply connection interface 310, an injection needle insertion button 312, and a slidable reagent insertion button 314. A vial 316 may connect to an upper portion of housing 302. Thermal energy probe 306 may be received in a hollow 318 in housing 302. Similarly, injection needle 308 may be received in a hollow 320 in housing 302. Thermal energy probe 306, injection needle 308, and energy supply connection interface 310 may be substantially the same as thermal energy probe 206, injection needle 208, and energy supply connection interface 210, respectively.

Dual applicator 300 may be used to treat a target tissue by applying thermal energy to the target tissue and applying a protein-crosslinker to the heat-shrunken target tissue. As described above, a distal end 322 of dual applicator 300 may be inserted through a minimally invasive incision to thereby contact the target tissue. Thermal energy probe 306 may be used to apply thermal energy to the target tissue. As described above with reference to thermal energy probe 206, thermal energy probe 306 may extend between a distal end 322 and a proximal end 324 in hollow 318 and may be longitudinally translatable therein by manipulating energy supply connection interface 310.

In some embodiments, injection needle 308 may be used to inject a protein crosslinker on or into the target tissue. Injection needle 308 may longitudinally translate in hollow 320 by actuating injection needle insertion button 312. Thus, in some embodiments, injection needle insertion button 312 may be used to control a position of injection needle 308. For example, injection needle insertion button 312 may be pushed toward distal end 322 such that injection needle 308 translates toward distal end 322. Similarly, injection needle insertion button 312 may be pulled toward proximal end 324 of dual applicator 300 such that injection needle 308 translates toward proximal end 324. The injection needle insertion button 312 may provide the same functionality as the trigger 212 of dual applicator 200 to thereby allow a user to adjust the depth of the injection needle 308 into the target tissue. A gauge 326 may provide a visual indication of the position of injection needle 308. In this way, the depth of injection needle 308 inside a target tissue may be adjusted and, as described above, injection needle 308 may be pulled out of the target tissue and distal end 322 may be positioned at a new location without having to remove dual applicator 300 from the minimally invasive incision. A depth stop 330 may limit the position of injection needle 308 when extended into the target tissue.

Vial 316 containing the protein crosslinker may be connected to the upper portion of housing 302. A seal on vial 316 may be penetrated by a needle in housing 302 to open the vial 316. In some embodiments, slidable reagent insertion button 314 may be used to draw a volume of protein crosslinker from Vial 316 into injection needle 308. For example, slidable reagent insertion button 314 may slide toward proximal end 324 such that a volume of protein crosslinker is drawn into injection needle 308. Slidable reagent insertion button 314 may be slidable toward distal end 322 to extrude the protein crosslinker from injection needle 308 into the target tissue. In some embodiments, slidable reagent insertion button 314 may draw a controlled volume of protein crosslinker from Vial 316 based on the distance that slidable reagent insertion button 314 is translated toward proximal end 324. For example, translating slidable reagent insertion button 314 completely toward proximal end 324 may draw a maximum volume of protein crosslinker from Vial 316. Similarly, translating slidable reagent insertion button 314 halfway toward proximal end 324 may draw a fractional volume of protein crosslinker from Vial 316. A gauge 328 may provide a visual indication of the volume of protein crosslinker drawn/extruded into/from the injection needle 308. In this way, a user of dual applicator 300 may control the dose of protein crosslinker injected into the target tissue.

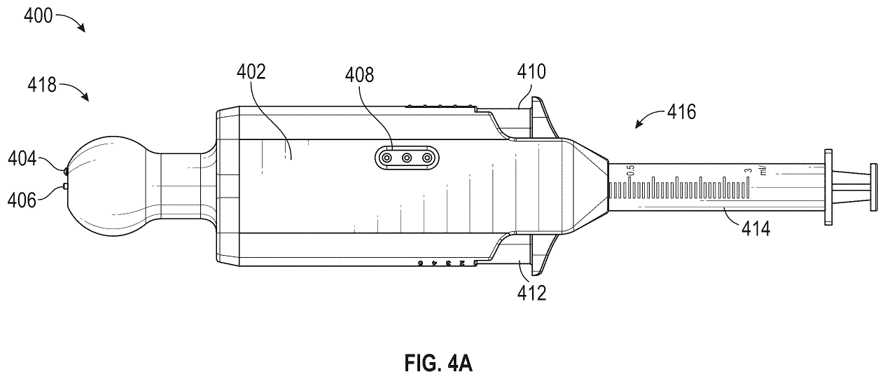

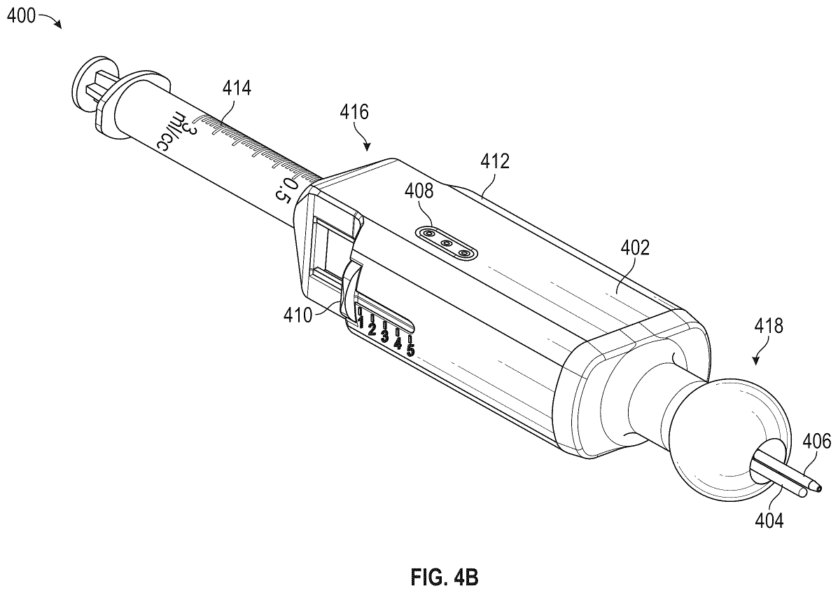

FIGS. 4A-B illustrate a third embodiment of a dual applicator 400 for use in performing the method of applying thermal heat to a target tissue and applying a protein-crosslinker to the heat-shrunken target tissue, such as method 100. Dual applicator 400 may comprise a housing 402, a thermal energy probe 404, an injection needle 406, an energy supply connection interface 408, a thermal energy probe insertion button 410, and an injection needle insertion button 412. A syringe vial 414 may be connected to a proximal end 416 of dual applicator 400. Thermal energy probe 404, injection needle 406, and syringe vial 414 may be substantially the same as thermal energy probe 206, injection needle 208, and syringe vial 216, respectively.

In some embodiments, dual applicator 400 may be used to treat a target tissue. For example, a minimally invasive incision may be made and a distal end 418 of dual applicator 400 may be inserted through the minimally invasive incision such that thermal energy probe 404 may contact the target tissue. In some embodiments, energy supply connection interface 408 may connect thermal energy probe 404 to an energy/power supply. In some embodiments, energy supply connection interface 408 is a standard plug. In some embodiments, energy supply connection interface 408 may be a metallic threaded connector. As described above, energy supply connection interface 408 may route power generated by the energy/power supply to thermal energy probe 404 such that thermal energy may be generated at the thermal energy probe 404. In some embodiments, energy/power routed to thermal energy probe may be regulated by a pre-programmed computer controller with adjustable inputs and switches such as a foot-operated switch or by a digital interface.

In some embodiments, thermal energy probe insertion button 410 may be used to control a position of thermal energy probe 404. For example, actuation of thermal energy probe insertion button 410 may control a position of thermal energy probe 404 within housing 402. For example, moving thermal energy probe insertion button 410 may translate thermal energy probe 404 within housing 402. In some embodiments, thermal energy probe insertion button 410 may be pushed/pressed toward distal end 418 such that thermal energy probe 404 translates forward past distal end 418, as shown in FIG. 4B. Similarly, when thermal energy probe insertion button 410 is pulled toward proximal end 416, thermal energy probe 404 may translate toward proximal end 416. Thus, when distal end 418 is inserted through a minimally invasive incision, the depth of thermal energy probe 404 in the minimally invasive incision may be adjusted by manipulating thermal energy probe insertion button 410. In this way, the thermal energy probe insertion button 410 may provide the same functionality as the energy supply connection interface 210, 310 manipulation to adjust a position of the thermal energy probe 206, 306, 404.

Injection needle insertion button 412 may be used to control a position of injection needle 406. Actuation of injection needle insertion button 412 may change the position of injection needle 406 within housing 402, thereby adjusting the depth of the injection needle 406 within the target tissue. For example, injection needle insertion button 412 may be pushed/pressed toward distal end 418 such that injection needle 406 translates past distal end 418, as shown in FIG. 4B. Similarly, when injection needle insertion button 412 is pulled toward proximal end 416, injection needle 406 may correspondingly translate toward proximal end 416. As such, depth of injection needle 406 in a minimally invasive incision may be adjusted by translating injection needle insertion button 412. As described above, such a feature is advantageous because, multiple injections of protein crosslinker may be administered to various areas of a target tissue without removing the distal end 418 of dual applicator 400 from the minimally invasive incision. In this way, injection needle insertion button 412 may provide the same functionality as the injection needle insertion button 312 and trigger 212 manipulation to thereby adjust a depth of the injection needle 208, 308, 406.

In some embodiments, injection needle 406 may be used to inject a protein crosslinker on or into a target tissue. For example, when distal end 418 is inserted through a minimally invasive incision, injection needle 406 may be used to inject protein crosslinker into the target tissue. As described above with respect to injection needle 208 and syringe vial 216, injection needle 406 and syringe vial 414 may function in substantially the same way.

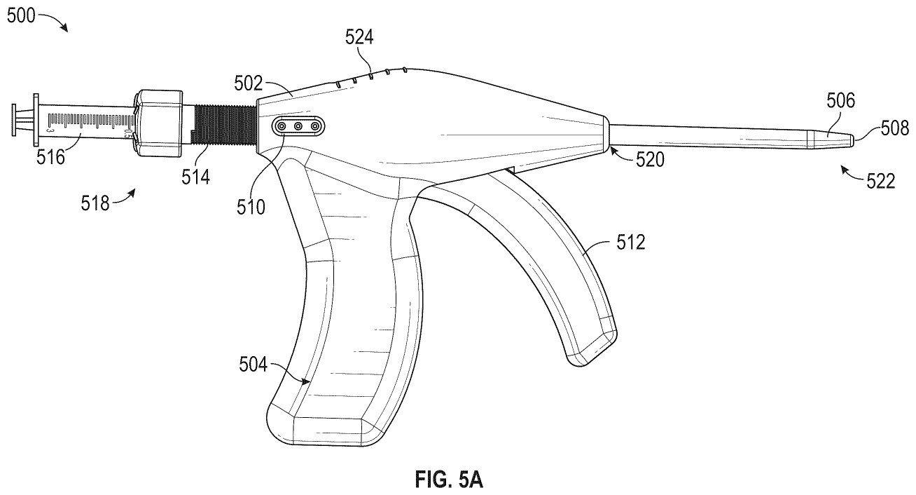

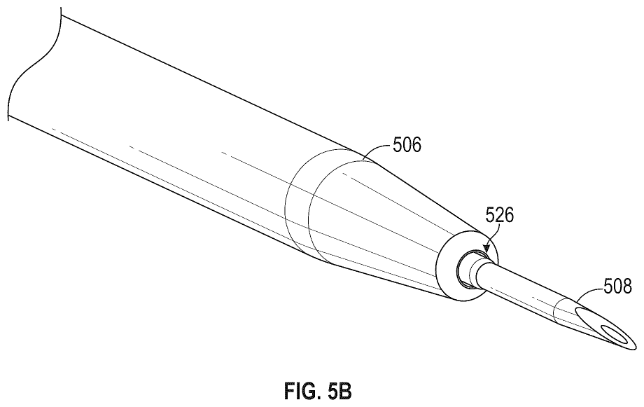

FIGS. 5A-5B illustrate a fourth embodiment of a dual applicator 500 for use in performing the method of applying thermal heat to a target tissue and applying a protein-crosslinker to the heat-shrunken target tissue, such as method 100. Dual applicator 500 may comprise a housing 502, a handle 504, a thermal energy probe 506, an injection needle 508, an energy supply connection interface 510, a trigger 512, a threaded connector 514, and a syringe vial 516 may be connected to a proximal end 518 of dual applicator 500. Thermal energy probe 506, injection needle 508, trigger 512, and syringe vial 516 may be substantially the same as thermal energy probe 206, injection needle 208, trigger 212, and syringe vial 216, respectively. Energy supply connection interface 510 may be substantially the same as energy supply connection interface 408. As such, housing 502 may comprise a hollow 520 in which thermal energy probe 506 is received, such that thermal energy probe 506 is longitudinally translatable in hollow 520. Hollow 520 may be substantially similar to hollow 226, 320.

In some embodiments, dual applicator 500 may be used to treat a target tissue. For example, a minimally invasive incision may be made and a distal end 522 of dual applicator 500 may be inserted through the minimally invasive incision such that thermal energy probe 506 and injection needle 508 may contact the target tissue. In some embodiments, trigger 512 may be used to control a position of thermal energy probe 506. Actuation of trigger 512 may adjust the depth of the thermal energy probe 506. For example, pulling trigger 512 toward proximal end 518 may cause thermal energy probe 506 to translate toward distal end 522. Pushing trigger 512 toward distal end 522 may cause thermal energy probe 506 to translate backward toward proximal end 518. A depth gauge 524 may be used to view the position of thermal energy probe 506. Depth gauge 524 may be substantially the same as depth gauge 220. In some embodiments, injection needle 508 may be a pop-out needle. In some embodiments, the intended position of injection needle 508 may be set using a dial, and insertion of the injection needle 508 as well as delivery of the protein crosslinker may be completed by a single push button on proximal end 518 of dual applicator 500.

As shown in FIG. 5B, thermal energy probe 506 may comprise a hollow 526 in which injection needle 508 is positioned. Hollow 526 may extend from proximal end 518 to distal end 522 of dual applicator 500. In this way, only a single structure comprising thermal energy probe 506 and injection needle 508 may require insertion through a minimally invasive incision to apply thermal energy and protein crosslinker to a target tissue.

Injection needle 508 may be translatable within thermal energy probe 506. For example, injection needle 508 may be translatable within hollow 526 of thermal energy probe 506. In some embodiments, threaded connector 514 may be used to control a position of injection needle 508. In some embodiments, actuation of threaded connector 514 may cause injection needle 508 to translate within thermal energy probe 506. For example, turning threaded connector 514 clockwise may cause injection needle 508 to translate through thermal energy probe 506 toward distal end 522, such that the depth of injection needle 508 through a minimally invasive incision and in a target tissue may be adjusted. As such, injection needle 508 may be pulled out of the target tissue and distal end 522 may be positioned at a new location without having to remove dual applicator 500 from the minimally invasive incision. In this way, multiple injections of protein crosslinker may be administered to various areas of a target tissue without removing the distal end 522 of dual applicator 500 from the minimally invasive incision. Threaded connector 514 may provide the same functionality as trigger 212, injection needle insertion button 312, and injection needle insertion button 412 to thereby adjust a position of the injection needle 208, 308, 406, 508 within housing 202, 302, 402, 502. In some embodiments, threaded connector 514 may be a threaded screw.

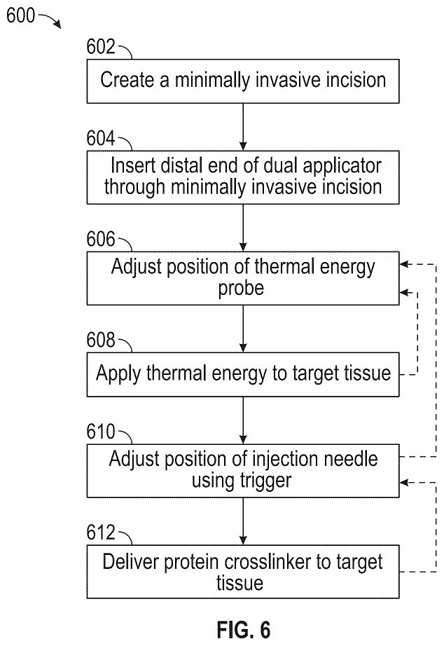

Turning now to FIG. 6, a method 600 of operating any of dual applicator 200, 300, 400, 500 to treat a target tissue is depicted. In step 602, a minimally invasive incision may be made to access a target tissue. In step 604, a distal end of a dual applicator, such as distal end 222, 322, 418, 522, may be inserted through the minimally invasive incision. In some embodiments, inserting a distal end of the dual applicator may comprise inserting a thermal energy probe, such as thermal energy probe 206, 306, 404, 506, and an injection needle, such as injection needle 208, 308, 406, 508, through the minimally invasive incision. In some embodiments, the thermal energy probe and the injection needle may be inserted through the same minimally invasive incision.

Step 606 may comprise adjusting a position of the thermal energy probe 206, 306, 404, 506 relative to the target tissue. In some embodiments, the thermal energy probe 206, 306, 404, 506 may be longitudinally translatable such that the probe may be translated forward to contact a target tissue. In some embodiments, step 606 may comprise translating the thermal energy probe 206, 306 using an energy supply connection interface 210, 310, described with reference to FIGS. 2A-B and 3. In some embodiments, step 606 may comprise translating the thermal energy probe 404 using a thermal energy insertion button 410, described with reference to FIGS. 4A-B. In some embodiments, step 606 may comprise translating the thermal energy probe 506 using a trigger 512, described with reference to FIGS. 5A-5B. In some embodiments, step 606 may comprise a user of the dual applicator adjusting the position of the thermal energy probe based on a distance from the thermal energy probe 206, 306, 404, 506 to the target tissue. For example, step 606 may comprise adjusting the position of thermal energy probe 206, 306, 404, 506 such that the thermal energy probe contacts the surface of the target tissue.

Step 608 may comprise applying thermal energy to the target tissue. In some embodiments, step 608 may comprise applying thermal energy to the target tissue while the thermal energy probe 206, 306, 404, 506 is in contact with the target tissue. In some embodiments, thermal energy is supplied to the thermal energy probe 206, 306, 404, 506 through an energy supply connection interface 210, 310, 408, 510. As described above, thermal energy routed to the thermal energy probe 206, 306, 404, 506 may be controlled by a pre-programmed computer controller with adjustable inputs and switches such as a foot-operated switch or by a digital interface. As such, step 608 may comprise causing thermal energy to be routed to the thermal energy probe 206, 306, 404, 506, and thereby to the target tissue, using a foot-operated switch or a digital interface. In some embodiments, step 608 may be substantially the same as step 106.

In some embodiments, method 600 may comprise performing step 606 before step 608 and then returning to step 606 after step 608. For example, step 608 may comprise applying thermal energy to a first region of a target tissue. Method 600 may then return to step 606 where a position of the thermal energy probe 206, 306, 404, 506 is adjusted. For example, step 606 may comprise translating the thermal energy probe 206, 306, 404, 506 toward a proximal end of the dual applicator, moving the thermal energy probe 206, 306, 404, 506 to a second region, then translating the thermal energy probe 206, 306, 404, 506 forward to thereby contact the second region of the target tissue. In some embodiments, steps 606 and 608 may comprise adjusting the position of the thermal energy probe and applying thermal energy to the target tissue multiple times in multiple regions of the target tissue, such that steps 606 and 608 comprising making multiple sweeps of the thermal energy probe and applying thermal energy in sweeps across the target tissue. Method 600 may then proceed from step 606 onward.

Step 610 may comprise adjusting a position of the injection needle 208, 308, 406, 508 relative to the target tissue. In some embodiments, the injection needle 208, 308, 406, 508 may be longitudinally translatable such that the injection needle may contact a target tissue. In some embodiments, step 610 may comprise translating the injection needle 208 using a trigger 212, described with reference to FIGS. 2A-B. In some embodiments, step 610 may comprise translating the injection needle 308, 406 using an injection needle insertion button 312, 412, described with reference to FIGS. 3 and 4. In some embodiments, step 610 may comprise translating the injection needle 508 using a threaded connector 514, described with reference to FIGS. 5A-5B. In some embodiments, step 610 may comprise a user of the dual applicator 200, 300, 400, 500 adjusting the position of the injection needle 208, 308, 406, 508 based on a distance from the injection needle 208, 308, 406, 508 to the target tissue. For example, step 610 may comprise adjusting the position of the injection needle 208, 308, 406, 508 such that the injection needle 208, 308, 406, 508 contacts the surface of the target tissue. In some embodiments, step 610 may comprise adjusting the position of the injection needle 208, 308, 406, 508 such that the injection needle 208, 308, 406, 508 penetrates the surface of the target tissue. For example, step 610 may comprise inserting the injection needle 208, 308, 406, 508 between 1 and 10 mm into the target tissue.

Step 612 may comprise delivering protein crosslinker to the target tissue. In some embodiments, protein crosslinker is delivered via the injection needle 208, 308, 406, 508. In some embodiments, step 612 may comprise actuating a syringe vial 216, 316, 414, 516 such that a liquid protein crosslinker is delivered via the injection needle 208, 308, 406, 508 to the target tissue. For example, syringe vial 216, 414, 516, respectively, may be actuated such that a liquid protein crosslinker injects into the target tissue through the injection needle 208, 406, 508. In some embodiments, step 612 may comprise actuating a slidable reagent insertion button 314, as described with reference to FIG. 3. For example, a slidable reagent insertion button 314 may be actuated toward a proximal end 324 of the dual applicator 300 to thereby draw a volume of the liquid protein crosslinker, then the slidable reagent insertion button 314 may be actuated toward a distal end of the dual applicator 300 to thereby release the liquid protein crosslinker into the target tissue through the injection needle 308, as described with reference to FIG. 3.

In some embodiments, method 600 may comprise performing steps 606-610 before step 612 then returning to step 610, then step 606 after performing step 612. For example, method 600 may comprise performing steps 606-612 on a first region of the target tissue. After step 610, method 600 may comprise returning to step 610 such that a position of the injection needle 208, 308, 406, 508 is adjusted by translating the injection needle 208, 308, 406, 508 toward a proximal end of the dual applicator, to thereby remove the injection needle 208, 308, 406, 508 from the target tissue. A user may then move the distal end of the dual applicator to a second region of the target tissue and proceed from step 606.

The order of operations depicted in FIG. 6 is not limiting such that each of steps 606-612 may be performed in any order. For example, in some embodiments, steps 606-612 may be performed sequentially. In some embodiments, steps 610, 612 may be performed before steps 606, 608. In some embodiments, steps 606, 610 may be performed before steps 608, 612. In some embodiments, steps 608, 612 are performed simultaneously.

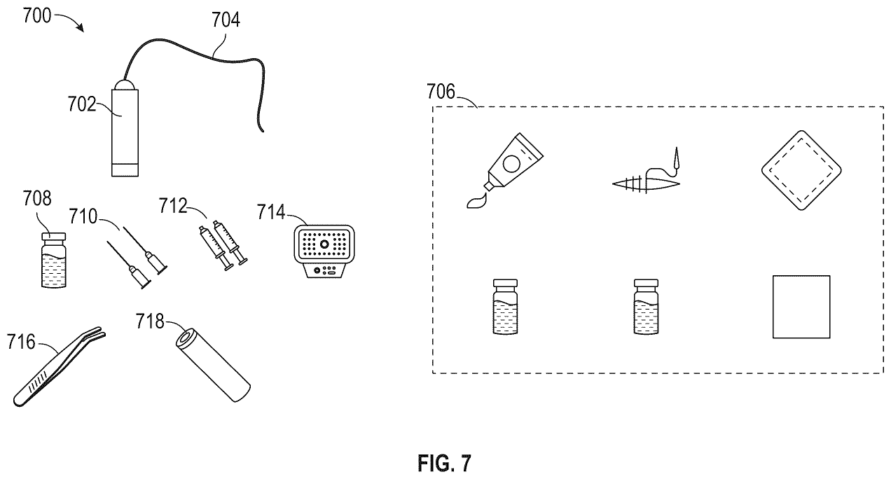

FIG. 7 illustrates a kit 700 (also referred to as a system) that may be used for treating a target tissue and performing the method of applying thermal energy to a target tissue and applying a protein crosslinker to the heat-shrunk target tissue, such as method 100 or method 600. In some embodiments, kit 700 may comprise a thermal energy device 702 with a wire 704 for connecting thermal energy device 702 to an energy/power supply/controller, protein crosslinker 706 (also referred to as protein crosslinker applicator 706), contrast agent 708, needles 710, syringes 712, mixer 714, forceps 716, and cannulated tube 718. Needles 710 may be of various gauges and shapes such that needles 710 are specific to a local anatomy of a target tissue. Syringes 712 may be various sizes such that a volume of liquid protein crosslinker to be injected is specific to a required dose for a target tissue.

In some embodiments, thermal energy device 702 may be a dual applicator 200, 300, 400, 500. In some embodiments, thermal energy device 702 may be an RF probe, such as a bipolar RF probe or a monopolar RF probe, or any other conventional thermal energy device.

In some embodiments, kit 700 may further comprise a protein crosslinker 706. Protein crosslinker 706 may be any of a dry powdered protein crosslinker in one vial with a buffered carrier in another vial, a microneedle patch containing protein crosslinker, a suture coated with protein crosslinker, a biodegradable film containing protein crosslinker, a biodegradable gel containing protein crosslinker or any combination thereof. Kit 700 may contain all of the aforementioned versions of protein crosslinker or may contain just one of the aforementioned versions of protein crosslinker.

In some embodiments, each type of protein crosslinker 706 in kit 700 may be used in a method, such as method 100, for treating a target tissue. For example, when protein crosslinker 706 is a dry powdered protein crosslinker with a buffered carrier, an injection reagent solution may be prepared and injected into a target tissue using needles 710 and syringes 712. In some embodiments, protein crosslinker 706 in kit 700 may be a genipin reagent comprising genipin within a range of 10 mM to 100 mM, prepared using the dry powdered protein crosslinker and buffered carrier. The genipin may be solubilized in a 4-(2-Hydroxyethyl)-1-piperazinepropanesulfonic acid (EPPS)-Phosphate aqueous buffer to form the genipin reagent, which may each be included as protein crosslinker 706 in kit 700. The EPPS-Phosphate aqueous buffer may comprise 50 mM to 500 mM phosphate ions and 50 mM to 250 mM EPPS buffered at a pH between 8.0 to 10.0. For example, the genipin reagent may contain 50 mM genipin in 50 mM EPPS buffer with 50 mM phosphate ions at pH 9. The concentration of genipin in the genipin reagent may be altered based upon a degree of laxity of the target tissue and within the confines of non-cytotoxic concentrations. In some embodiments, any type of aqueous buffer may be used to solubilize the genipin into a genipin reagent.

In some embodiments, the protein crosslinker 706 (e.g., genipin) in kit 700 may be injected into the target tissue through the minimally invasive incision using needles 710 and syringes 712. In some embodiments, the genipin may be applied topically on a surface of the target degraded tissue. The genipin reagent solution may be applied topically to a surface of the target tissue. In some embodiments, the genipin reagent is injected in the target tissue and/or topically on the target tissue during thermal shrinkage. In some embodiments, the genipin solution may be injected and/or topically applied to the target tissue after thermal shrinkage. For example, the genipin reagent may be injected directly into the thermally shrunk tissue and/or applied topically on the thermally shrunk tissue. In some embodiments, the genipin reagent may be injected and/or applied topically to the target tissue before, during, or after thermal shrinkage via the thermal energy device 702, such as by using dual applicator 200, 300, 400, 500. In some embodiments, when protein crosslinker 706 is a dry powdered protein crosslinker with a buffered carrier, step 108 of method 100 may be carried out using a fluoroscopy-guided injection. In such an instance, contrast agent 708 may be used in addition to a liquid protein crosslinker prepared from the dry powdered protein crosslinker and buffered carrier. Contrast agent 708 may be an iodinated compound.

In some embodiments, protein crosslinker 706 in kit 700 may be a microneedle patch which may be used in method 100, wherein step 108 may comprise applying a degradable microneedle patch to the target tissue immediately after thermal shrinkage via thermal energy device 702, such as a bipolar RF probe or any of dual applicator 200, 300, 400, 500. Each needle of the microneedle patch may contain the protein crosslinker. Thermal energy device 702 may deliver thermal energy to target tissue, and the microneedle patch containing a protein crosslinker may cover the treated tissue shortly after thermal energy application. The microneedle patch may deliver the genipin over time and in a controlled manner. Thermal energy device 702, such as an RF probe or any of dual applicator 200, 300, 400, 500, may access the target tissue via a small incision. The microneedle patch may be applied to the target tissue using forceps 716. In some embodiments, the microneedle patch may be applied to the target tissue through a cannulated tube 718. In some embodiments, the microneedle patch may be adhesive on the side of the microneedles to stabilize the patch to the target tissue after rolling it up and inserting it through the cannulated tube 718. When the adhesive side of the microneedle patch is placed on target tissue, it may unroll and stick to the target tissue for localized and controlled delivery. The microneedle patch may be adhesive to remain localized to the target region of the tissue. The protein-crosslinker-filled microneedles may slightly penetrate the tissue, fixating the patch to the target tissue and releasing genipin deeper than the surface of the target tissue. The microneedle patch may be cooled prior to application to prevent collagen denaturation and damage from the thermal energy application. The microneedle patch may also be sprayed with a cooling cryogen spray immediately prior to patch application on the target tissue. The microneedle patch may be degradable to avoid an explantation procedure and for gradual delivery. The microneedles may be coated in an antibiotic or an anti-inflammatory agent.

In some embodiments, protein crosslinker 706 in kit 700 may be a suture coated with protein crosslinker, a degradable suture containing protein crosslinker, or a degradable suture containing protein crosslinker that is also coated with protein crosslinker, which may be used in method 100, wherein step 108 may comprise applying a said suture to the target tissue immediately after thermal shrinkage via thermal energy device 702, such as a bipolar RF probe, or any of dual applicator 200, 300, 400, 500. For example, in steps 102 and 104, a surgeon may create an incision through the skin and subcutaneous tissues to gain access to the target degraded tissue (e.g., tendon, ligament). In step 106, the thermal energy device 702, such as dual applicator 200, 300, 400, 500, or a bipolar RF probe, may be inserted into the incision to apply heat energy to the target tissue (i.e., contacting the surface of the target tissue). Once the tissue has been shrunk the desired amount, in step 108 the surgeon may remove the thermal energy device 702 from the incision and apply a suture with a degradable coating containing protein crosslinker 706 (e.g. genipin) and a plasticizer to the target tissue or to adjacent tissues such that the suture with the degradable coating contacts the target tissue. In some embodiments, forceps 716 of kit 700 may be used to apply the protein crosslinker 706 (e.g., a suture coated with protein crosslinker). As the coating degrades, the protein crosslinker 706 may be absorbed and/or otherwise be released into the target tissue. The surgeon may close the incision using a suture or the suture with the degradable coating. In some embodiments, the suture (or other implantable device) coated or otherwise containing protein crosslinker 706 may be inserted into the target tissue, thereby releasing the protein crosslinker 706 to the inside of the target tissue.

In some embodiments, protein crosslinker 706 in kit 700 may be a biodegradable film containing protein crosslinker 706 which may be used in method 100. For example, step 108 may comprise delivering a protein crosslinker 706, such as genipin to the target tissue through the biodegradable film applied to the external surface of the target tissue either before, during, or after shrinking with thermal energy. A degradable film comprising a protein crosslinker 706 may further comprise two layers. For example, the degradation film may comprise a first layer containing the protein crosslinker 706 that contacts the target tissue and a second, top layer that may be a barrier to the release of crosslinker 706 outside of the target tissue. For example, a small incision may be made above a target tendon to allow for a two layered degradable film containing the protein crosslinker genipin to be applied to the tissue. The film may degrade over a period of time and release the protein crosslinker 706 into the target tissue across the surface of the target tissue in contact with the biodegradable film.

In some embodiments, protein crosslinker 706 of kit 700 may be a biodegradable gel containing protein crosslinker 706 which may be used in method 100. For example, step 108 may comprise delivering a protein crosslinker 706, such as genipin, to the target tissue through a biodegradable gel applied to the external surface of the target tissue either before, during, or after shrinking with thermal energy. The biodegradable gel may be applied or painted on the target tissue to deliver a protein crosslinker 706 locally over time. For example, a tissue may be accessed via a minimally invasive incision and thermal energy device 702, such as an RF probe or any of dual applicator 200, 300, 400, 500, used to apply thermal energy to shrink the target tissue. To mechanically reinforce the target tissue after shrinking, protein crosslinker 706, such as genipin, may be applied by painting a biodegradable gel onto the previously shrunk target tissue.

Further, kit 700 may comprise mixer 714, which may be any one of an ultrasonic homogenizer, a vortex mixer, an orbital shaker, a rocking platform, and an inversion mixer. Mixer 714 may be used to prepare a liquid protein crosslinker. For example, protein crosslinker 706 may come as a vial of dried protein crosslinker and a vial of buffered carrier that is to be mixed with the dried protein crosslinker to prepare the liquid protein crosslinker. In some embodiments, mixer 714 may be used to aid in solubilizing the dried protein crosslinker in the buffered carrier by mechanically agitating the solution.

Kit 700 may comprise any and all of the aforementioned components, including various combinations thereof that may be specific to the planned procedure and the anatomy of the target tissue to be treated.

An exemplary experiment showing the immediate effects of a protein crosslinker on target tissue subject to thermal shrinkage is discussed below.

To demonstrate the capability of a genipin treatment to immediately increase the strength and improve other material properties of tendon tissue following a thermal treatment producing a 15% to 20% shrinkage of the tissue, bovine Achilles tendons were subjected to controlled thermal heating to induce collagen denaturation and thermal shrinkage. The bovine Achilles tendons were subjected to a thermal bath for 12 minutes at 60° C. to induce collagen denaturation and thermal shrinkage. After thermal shrinkage, half of the shrunk tendon specimens were immersed for 16 hours in a 20 mM genipin isotonic phosphate buffered saline (PBS) bath overnight to ensure maximum delivery of genipin to the shrunk tissue. The other shrunk tendon specimens and the control (not shrunk) specimens were also immersed in a genipin-free isotonic PBS bath for 16 hours prior to mechanical testing.

Specimens from each experimental group were then cut into hour-glass shaped specimens, mid-substance cross-sectional areas were measured, and each specimen was wrapped in saline soaked gauze to prevent dehydration prior to testing. Each specimen was fixed in a materials testing machine and then subjected to cyclic loading (10 cycles to 5% strain), stress relaxation (2 minutes at 10% strain), and then loaded to failure.

Load-history dependence (a viscoelastic characteristic) was determined by the difference in peak stress from the first to the tenth 5% strain loading cycle. Hysteresis (energy absorbed during a loading-unloading cycle) was calculated for the 10th loading cycle. Stress-relaxation was determined by the drop in stress during the 2-minute hold at 10% strain. The elastic-plastic characteristics that were measured included toe-region size, elastic region Young's modulus, yield stress, yield strain, ultimate tensile stress, ultimate tensile strain, resilience (energy to yield point), and toughness (energy to full failure).

Data analysis from these experiments showed mean ultimate tensile stress (UTS) of 16.8 MPa for the control group, 10.4 MPa for the shrunk-untreated specimens, and 15.8 MPa for the shrunk-genipin treated group. The shrunk specimens' strength was 38% below that of the control group, and the shrunk and genipin treated specimens' strength was 6% below the control strength.

Likewise, mean strain at the ultimate tensile stress point was 15.6 for the control group, 19.2 for the shrunk-untreated specimens, and 15.9 for the shrunk-genipin treated group. The shrunk group's strain at peak tensile load was 23% greater than the control group, and the shrunk and genipin treated group strain at peak tensile load was 2% greater than the strain at peak tensile load of the control group. Likewise, the shrunk-genipin treated specimens demonstrated a restoration of yield stress and strain, elastic modulus, resilience, toughness, hysteresis, and stress relaxation compared to the shrunken group.

The results demonstrate a genipin treatment is capable of immediately restoring material properties, such as tensile strength and appropriate levels of strain, and viscoelastic properties, to shrunk, heat denatured and disrupted collagenous tissue, such as tendon specimens. Material properties, such as tensile strength and appropriate levels of strain may be restored back to the pre-heat-shrinking-treatment levels. Thus, genipin treatment may restore mechanical integrity and material properties of thermally shrunk tissue back to pre-treatment levels, avoiding the need for weeks of joint immobilization or loading avoidance to promote tissue healing and regeneration to partially restore mechanical properties.

Features described above, as well as those claimed below, may be combined in various ways without departing from the scope hereof. The follow examples illustrate some possible, non-limiting combinations:

Clause 1. A method of delivering a protein crosslinker to a target tissue at a treatment site, the method comprising: creating a minimally invasive incision at the treatment site; inserting a thermal energy device into the minimally invasive incision; applying thermal energy to the target tissue using the thermal energy device; and contacting the shrunk target tissue with a protein crosslinker to thereby restore a mechanical property of the shrunk target tissue, such that a mechanical integrity of the shrunk target tissue is improved.

Clause 2: The method of clause 1, wherein applying thermal energy further comprises contacting at least a portion of the target tissue with the thermal energy device to shrink the target tissue.

Clause 3. The method of clause 1 or clause 2, wherein contacting at least a portion of the shrunk target tissue with the protein crosslinker comprises injecting the protein crosslinker into the shrunk target tissue.

Clause 4. The method of any of clauses 1-3, wherein the protein crosslinker is genipin and the genipin is solubilized in a buffer to form a genipin reagent, the genipin reagent containing genipin within a range of 10 mM to 100 mM.

Clause 5. The method of any of clauses 1-4, wherein the buffer contains 50 mM to 500 mM phosphate ions and 50 mM to 250 mM 4-(2-Hydroxyethyl)-1-piperazinepropanesulfonic acid, wherein the genipin reagent is buffered at a pH between 8.0 and 10.0.

Clause 6. The method of any of clauses 1-5, wherein the mechanical property is at least one of strength, fatigue resistance, stress-relaxation, creep, Young's modulus, yield stress, yield strain, ultimate tensile stress, ultimate tensile strain, resilience, toughness, load-history dependence, hysteresis, storage and loss moduli.

Clause 7. The method of any of clauses 1-6, wherein the thermal energy device defines a proximal end, a distal end, and a hollow extending from the distal end to the proximal end, wherein contacting the shrunk target tissue with the protein crosslinker further comprises: injecting the protein crosslinker through the hollow.

Clause 8. The method of any of clauses 1-7, wherein contacting the shrunk target tissue with the protein crosslinker comprises placing at least one of a microneedle patch, a biodegradable film, a biodegradable gel, a protein crosslinker-coated suture, or a protein crosslinker containing degradable suture on the shrunk target tissue.

Clause 9. The method of any of clauses 1-8, wherein the thermal energy device comprises a bipolar radiofrequency probe having at least one active electrode and at least one return electrode.

Clause 10. A dual applicator for treating a target tissue comprising: a housing; a thermal energy probe received in the housing; an energy supply connection interface coupled to the thermal energy probe and configured to supply thermal energy to the thermal energy probe; and an injection needle comprised in the housing.

Clause 11. The dual applicator of clause 10, wherein the thermal energy probe is longitudinally translatable within the housing.

Clause 12. The dual applicator of clause 10 or 11, wherein the injection needled is coupled to a trigger.

Clause 13: The dual applicator of any of clauses 10-12, wherein actuating the trigger causes longitudinal translation of the injection needle to thereby adjust a depth of the injection needle inserted into the target tissue.

Clause 14. The dual applicator of any of clauses 10-13, further comprising a Luer lock connector operable to receive a syringe vial.

Clause 15. The dual applicator of any of clauses 10-14, further comprising a depth gauge, wherein the depth gauge indicates a position of the injection needle.

Clause 16. The dual applicator of any of clauses 10-15, further comprising a depth stop, wherein the depth stop limits the position of the injection needle when extended into the target tissue.

Clause 17. The dual applicator of any of clauses 10-16, wherein actuating the energy supply connection interface causes longitudinal translation of the thermal energy probe.

Clause 18. The dual applicator of any of clauses 10-17, further comprising an aperture, wherein the aperture provides a view of the syringe vial connected to the Luer lock connector.

Clause 19. The dual applicator of any of clauses 10-18, wherein the thermal energy probe is a bipolar radiofrequency probe comprising at least one active electrode and at least one return electrode.

Clause 20. The dual applicator of any of clauses 10-19, wherein the injection needle is received through a hollow in the housing.

Clause 21. A method of treating a target tissue, comprising: creating a minimally invasive incision; inserting, through the minimally invasive incision, a distal end of a dual applicator, wherein the dual applicator comprises: a thermal energy probe; an injection needle; and a vial containing a protein crosslinker; adjusting a position of the thermal energy probe relative to the target tissue; applying thermal energy to a first region via the thermal energy probe; adjusting a position of the injection needle relative to the target tissue; and delivering the protein crosslinker to the first region from the vial.

Clause 22. The method of clause 21, wherein the dual applicator comprises a trigger.

Clause 23. The method of clause 21 or 22, wherein the adjustment comprises contacting a first region of the target tissue with the thermal energy probe.

Clause 24. The method of any of clauses 21-23, wherein the adjustment of the injection needle comprises: inserting the injection needle into the first region; and actuating the trigger to translate the injection needle within the dual applicator.

Clause 25. The method of any of clauses 21-24, further comprising: performing a second adjustment of the position of the injection needle, wherein the second adjustment of the position of the injection needle comprises inserting the injection needle into a second region by actuating the trigger to translate the injection needle out of the first region and into the second region; and delivering the protein crosslinker to the second region from the vial.

Clause 26. The method of any of clauses 21-25, wherein applying thermal energy to the first region comprises applying a voltage of between 10 volts to 500 volts to the first region.

Clause 27. The method of any of clauses 21-26, wherein applying thermal energy to the first region comprises applying thermal energy to the first region until the first region reaches a predefined temperature.

Clause 28. The method of any of clauses 21-27, wherein applying thermal energy to the first region further comprises: applying thermal energy to the first region of the target tissue until the target tissue has shrunk by a range of 10% to 50% of a pre-treatment length of the target tissue.

Clause 29. The method of any of clauses 21-28, wherein the dual applicator further comprises an energy supply connection interface coupled to the thermal energy probe and configured to supply thermal energy to the thermal energy probe, wherein adjusting the position of the thermal energy probe further comprises moving the energy supply connection interface to thereby translate the thermal energy probe.

Clause 30. A dual applicator, comprising: a housing; a thermal energy probe; an injection needle; a trigger; a threaded connector; a vial, containing a protein crosslinker.

Clause 31. The dual applicator of clause 30, wherein the thermal energy probe is positioned through a first hollow in the housing.

Clause 32. The dual applicator of clause 30 or clause 32, wherein the injection needle is positioned through a second hollow in the thermal energy probe.

Clause 33. The dual applicator of any of clauses 30-32, wherein a position of the thermal energy probe is controlled by the trigger.

Clause 34. The dual applicator of any of clauses 30-33, wherein a position of the injection needle is controlled by the threaded connector.

Clause 35. The dual applicator of any of clauses 30-34, further comprising an energy supply connection interface.

Clause 36. The dual applicator of any of clauses 40-35, wherein the energy supply connection interface supplies energy to the thermal energy probe.

Clause 37. The dual applicator of any of clauses 30-36, wherein the thermal energy probe is a bipolar radiofrequency probe comprising at least one active electrode and at least one return electrode.

Clause 38. The dual applicator of any of clauses 30-37, wherein the injection needle is longitudinally translatable in the second hollow.

Clause 39. The dual applicator of any of clauses 30-38, further comprising a depth gauge, wherein the depth gauge indicates a position of the thermal energy probe.

Clause 40. The dual applicator of any of clauses 30-39, further comprising a Luer lock connector operable to receive a syringe vial.

Clause 41. A system for treating a target tissue, comprising: a thermal energy probe; a wire, wherein the wire connects the thermal energy probe to power supply; a protein crosslinker applicator, wherein the protein crosslinker applicator comprises at least one of a dry powdered protein crosslinker, a microneedle patch, a biodegradable film, a biodegradable gel, a suture coated with protein crosslinker, a biodegradable suture containing a protein crosslinker; at least one needle; at least one syringe.

Clause 42. The system of clause 41, further comprising a cannulated tube.

Clause 43. The system of clause 41 or clause 42, further comprise at least one pair of forceps.

Clause 44. The system of any of clauses 40-43, further comprising a mixer.

Clause 45. The system of any of clauses 41-44, further comprising a contrast agent.

Clause 46. The system of any of clauses 41-45, wherein the thermal energy probe is a bipolar radiofrequency probe.

Clause 47. The system of any of clauses 41-46, further comprising a buffered carrier.

1. A method of delivering a protein crosslinker to a target tissue at a treatment site, the method comprising:
creating a minimally invasive incision at the treatment site;
inserting a thermal energy device into the minimally invasive incision;
applying thermal energy to the target tissue using the thermal energy device,
wherein applying thermal energy further comprises contacting at least a portion of the target tissue with the thermal energy device to shrink the target tissue; and
contacting the shrunk target tissue with a protein crosslinker to thereby restore a mechanical property of the shrunk target tissue, such that a mechanical integrity of the shrunk target issue is improved.

2. The method of claim 1,
wherein contacting at least a portion of the shrunk target tissue with the protein crosslinker comprises injecting the protein crosslinker into the shrunk target tissue.

3. The method of claim 2,
wherein the protein crosslinker is genipin and the genipin is solubilized in a buffer to form a genipin reagent;
wherein the genipin reagent contains genipin within a range of 10 mM to 100 mM;
wherein the buffer contains 50 mM to 500 mM phosphate ions and 50 mM to 250 mM 4-(2-Hydroxyethyl)-1-piperazinepropanesulfonic acid; and
wherein the genipin reagent is buffered at a pH between 8.0 and 10.0.

4. The method of claim 1, wherein the mechanical property is at least one of strength, fatigue resistance, stress-relaxation, creep, Young's modulus, yield stress, yield strain, ultimate tensile stress, ultimate tensile strain, resilience, toughness, load-history dependence, hysteresis, storage and loss moduli.

5. The method of claim 1,
wherein the thermal energy device defines a proximal end, a distal end, and a hollow extending from the distal end to the proximal end,
wherein contacting the shrunk target tissue with the protein crosslinker further comprises:
injecting the protein crosslinker through the hollow.

6. The method of claim 1, wherein contacting the shrunk target tissue with the protein crosslinker comprises placing at least one of a microneedle patch, a biodegradable film, a biodegradable gel, or a protein crosslinker-coated suture on the shrunk target tissue.

7. The method of claim 1,
wherein the thermal energy device comprises a bipolar radiofrequency probe having at least one active electrode and at least one return electrode.

8. A dual applicator for treating a target tissue comprising:
a housing;
a thermal energy probe received in the housing,
wherein the thermal energy probe is longitudinally translatable within the housing;
an energy supply connection interface coupled to the thermal energy probe and configured to supply thermal energy to the thermal energy probe; and
an injection needle comprised in the housing and coupled to a trigger;
wherein actuating the trigger causes longitudinal translation of the injection needle to thereby adjust a depth of the injection needle inserted into the target tissue.

9. The dual applicator of claim 8, further comprising a Luer lock connector operable to receive a syringe vial.

10. The dual applicator of claim 8, further comprising a depth gauge, wherein the depth gauge indicates a position of the injection needle.

11. The dual applicator of claim 8, wherein actuating the energy supply connection interface longitudinal translates the thermal energy probe.

12. The dual applicator of claim 9, further comprising an aperture, wherein the aperture provides a view of the syringe vial connected to the Luer lock connector.

13. The dual applicator of claim 8, wherein the thermal energy probe is a bipolar radiofrequency probe comprising at least one active electrode and at least one return electrode.

14. The dual applicator of claim 8, wherein the injection needle is received through a hollow in the housing.

15. A method of treating a target tissue, comprising:
creating a minimally invasive incision,
inserting, through the minimally invasive incision, a distal end of a dual applicator, wherein the dual applicator comprises:
a thermal energy probe;
an injection needle;
a trigger; and
a vial containing a protein crosslinker;
adjusting a position of the thermal energy probe relative to the target tissue,
wherein the adjustment comprises contacting a first region of the target tissue with the thermal energy probe;
applying thermal energy to the first region via the thermal energy probe,
adjusting a position of the injection needle relative to the target tissue,
wherein the adjustment of the injection needle comprises:
inserting the injection needle into the first region; and
actuating the trigger to translate the injection needle within the dual applicator; and
delivering the protein crosslinker to the first region from the vial.

16. The method of claim 15, further comprising:
performing a second adjustment of the position of the injection needle,
wherein the second adjustment of the position of the injection needle comprises inserting the injection needle into a second region by actuating the trigger to translate the injection needle out of the first region and into the second region; and
delivering the protein crosslinker to the second region from the vial.

17. The method of claim 15, wherein applying thermal energy to the first region comprises applying a voltage within a range of 10 volts to 500 volts to the first region.

18. The method of claim 15, wherein applying thermal energy to the first region comprises applying thermal energy to the first region until the first region reaches a predefined temperature.

19. The method of claim 15, wherein applying thermal energy to the first region further comprises:
applying thermal energy to the first region of the target tissue until the target tissue has shrunk by within a range of 10% to 50% of a pre-treatment length of the target tissue.

20. The method of claim 15, wherein the dual applicator further comprises an energy supply connection interface coupled to the thermal energy probe and configured to supply thermal energy to the thermal energy probe,
wherein adjusting the position of the thermal energy probe further comprises moving the energy supply connection interface to thereby translate the thermal energy probe.
